(19) United States
(12) Patent Application Publication
(10) Pub. No.: US 2021/0171899 A1
(43) Pub. Date: Jun. 10, 2021

(54) METHODS AND PRODUCTS FOR BIODEGRADATION OF WASTE

(71) Applicant: Westcom Solutions Pte Ltd, Singapore (SG)

(72) Inventors: Juturu Veeresh, Singapore (SG); Jinchuan Wu, Singapore (SG); David Tan, Singapore (SG); Ruby Fang, Singapore (SG)

(73) Assignee: Westcom Solutions Pte Ltd, Singapore (SG)

(21) Appl. No.: 16/769,402

(22) PCT Filed: May 25, 2018
(86) PCT No.: PCT/SG2018/050259

§ 371 Date: Jun. 3, 2020

(30) Foreign Application Priority Data
Dec. 6, 2017 (SG) 10201710123W

(51) Int. Cl. C12N 1/20; C12N 1/16; C12N 11/02; C02F 3/34; B09B 3/00

(52) U.S. Cl. CPC C12N 1/20; C12N 1/16; B09B 3/00; C02F 3/341; C02F 3/347; C12N 11/02

(57) ABSTRACT

The present invention provides a method of producing a product for waste degradation, a product for waste degradation, an isolated microbial strain, a substantially pure culture of the isolated microbial strain, a microbial consortium or a mixed microbial composition, and a waste degradation method. The method of producing a product for waste degradation includes: isolating a plurality of microbial strains from one or more sources of food waste as separate colonies on a solid medium; selecting a plurality of the isolated microbial strains based on size and/or abundance of colonies of each of the microbial strains, the selected microbial strains being selected based on having a larger colony size and/or a higher colony abundance compared to other microbial strains; and combining the selected microbial strains to produce a microbial consortium for waste degradation.

### FIELD OF THE INVENTION

The present invention relates to the field of waste biodegradation. In particular, the present invention relates to the field of biodegradation of waste comprising organic matter; utilising microorganisms to efficiently biodegrade the waste.

### BACKGROUND OF THE INVENTION

The amount of waste generated by the human population continues to increase at an alarming rate. Almost any human activity would generate waste. Such waste can be in the solid, liquid or gaseous form. Waste is generated by any living organism. The simple act of feeding by a higher living organism inevitably generates waste as the organism may not consume the entire food portion and the consumed food is not completely digested and absorbed.

Human waste includes municipal waste, sewage waste and industrial waste for example. Proper waste management is of increasing importance and there are many factors, aspects and goals to consider; such as reduction/removal of toxic and hazardous materials, the familiar reducing, reusing and recycling slogan. Biodegradation of organic waste is an important arm of the waste management spectrum and it is therefore desirable to develop new and improved biodegradation processes as part of combatting the waste problem.

### SUMMARY OF THE INVENTION

Accordingly, in a first aspect, the present invention provides a method of producing a product for waste degradation. The method includes isolating a plurality of microbial strains from one or more sources of food waste as separate colonies on a solid medium; selecting a plurality of the isolated microbial strains based on size and/or abundance of colonies of each of the microbial strains, the selected microbial strains being selected based on having a larger colony size and/or a higher colony abundance compared to other microbial strains; and combining the selected microbial strains to produce a microbial consortium for waste degradation.

In a second aspect, the present invention provides a product for waste degradation including a microbial consortium. The microbial consortium includes a combination of microbial strains isolated from one or more sources of food waste as separate colonies on a solid medium and selected based on size and/or abundance of colonies of each of the microbial strains, the selected microbial strains being selected based on having a larger colony size and/or a higher colony abundance compared to other microbial strains.

In a third aspect, the present invention provides an isolated microbial strain selected from a group consisting of Candida sp., Pediococcus sp. and Enterobacter cloacae. In particular, the group may consist of Candida glabrata DSMZ 32770, Pediococcus sp. DSMZ 32729, Enterobacter cloacae DSMZ 32739 and Enterobacter sp. DSMZ 32730. The DSMZ numbers are the deposit numbers with Leibniz-Institute DSMZ-German Collection of Microorganisms (a Budapest treaty international deposit authority).

In a fourth aspect, the present invention provides a substantially pure culture of any of the isolated microbial strains in accordance with the third aspect.

In a fifth aspect, the present invention provides a microbial consortium or a mixed microbial composition comprising two or more strains selected from a group consisting of Candida sp., Pediococcus sp. and Enterobacter cloacae. In particular, the group may consist of Candida glabrata DSMZ 32770, Pediococcus sp. DSMZ 32729, Enterobacter cloacae DSMZ 32739 and Enterobacter sp. DSMZ 32730.

In a sixth aspect, the present invention provides a waste degradation method. The method includes providing one of the product for waste degradation in accordance with the second aspect, the isolated microbial strain in accordance with the third aspect, the substantially pure culture of the isolated microbial strain in accordance with the fourth aspect and the microbial consortium or the mixed microbial composition in accordance with the fifth aspect; mixing the one of the product for waste degradation, the isolated microbial strain, the substantially pure culture of the isolated microbial strain and the microbial consortium or the mixed microbial composition with waste; and biodegrading the waste with the one of the product for waste degradation, the isolated microbial strain, the substantially pure culture of the isolated microbial strain and the microbial consortium or the mixed microbial composition.

Other aspects and advantages of the invention will become apparent from the following detailed description, taken in conjunction with the accompanying drawings, illustrating by way of example the principles of the invention.

### BRIEF DESCRIPTION OF THE DRAWINGS

FIG. 1 is a photograph showing a comparison between immobilized microbial cells in accordance with an embodiment of the present invention before (0 h) and after (72 h) air drying;

FIG. 2 is a photograph showing chicken waste before (0 h) and after (162 h) degradation using a waste degradation product in accordance with an embodiment of the present invention;

FIG. 3 is a photograph showing cooked meat before (0 h) and after (114 h) degradation using a waste degradation product in accordance with an embodiment of the present invention;

FIG. 4 is a photograph showing fruit waste including bagasse before (0 h) and after (114 h) degradation using a waste degradation product in accordance with an embodiment of the present invention;

FIG. 5 is a photograph showing a waste degradation product in accordance with an embodiment of the present invention prepared by co-cultivation of four (4) microbial strains and immobilized on saw dust;

FIGS. 6(a) through 6(c) are photographs showing (a) cooked meat, (b) degraded meat at 0 h and (c) degraded meat after 48 h using a waste degradation product in accordance with an embodiment of the present invention;

FIGS. 7(a) through 7(c) are photographs showing (a) cooked meat, (b) degraded meat at 0 h and (c) degraded meat after 48 h using a commercial microbial consortium;

FIGS. 8(a) through 8(c) are photographs showing (a) combined noodles and meat, (b) degraded products at 0 h and (c) degraded products after 48 h using a waste degradation product in accordance with an embodiment of the present invention;

FIGS. 9(a) through 9(c) are photographs showing (a) combined noodles and meat, (b) degraded products at 0 h and (c) degraded products after 48 h using a commercial microbial consortium;

FIGS. 10(a) through 10(c) are photographs showing (a) noodles, (b) degraded noodles at 0 h and (c) degraded noodles after 120 h using a waste degradation product in accordance with an embodiment of the present invention; and

FIGS. 11(a) through 11(c) are photographs showing (a) noodles, (b) degraded noodles at 0 h and (c) degraded noodles after 120 h using a commercial microbial consortium.

### DEFINITIONS

As used herein, “biodegrade” means to break down or decompose by biological processes. Thus, the process of decomposing an organic material by contacting the material with bacteria is an example of biodegradation.

As used herein, the term “comprising” or “including” is to be interpreted as specifying the presence of the stated features, integers, steps or components as referred to, but does not preclude the presence or addition of one or more features, integers, steps or components, or groups thereof. However, in context with the present disclosure, the term “comprising” or “including” also includes “consisting of”. The variations of the word “comprising”, such as “comprise” and “comprises”, and “including”, such as “include” and “includes”, have correspondingly varied meanings.

As used herein, “microorganism” is understood to refer to any microscopic organism including a eukaryote, a prokaryote or a virus; further including, but not limited to, a bacterium (either gram positive, gram negative or gram− variable), a fungus, a virus, a protozoan, algae and reproductive forms thereof including cysts and spores. With respect to bacteria, for example, it encompasses mycoplasmas, rickettsiae, and chlamydiae, which replicate within eukaryotic cells, as well as those bacteria which do not. The term “microorganism” may be used interchangeably with “microbial organism” and “microbe”.

As used herein, the term “isolated” as applied to a microorganism refers to a microorganism which has been removed and/or purified from an environment in which it naturally occurs. As such, an “isolated strain” of a microbe as used herein is a strain that has been removed and or purified from its natural milieu. Thus, an “isolated microorganism” does not include one residing in an environment in which it naturally occurs. Further, the term “isolated” does not necessarily reflect the extent to which the microbe has been purified. A “substantially pure culture” of the strain of microbe refers to a culture which contains substantially no other microbes than the desired strain or strains of microbe. In other words, a substantially pure culture of a strain of microbe is substantially free of other contaminants, which can include microbial contaminants as well as undesirable chemical contaminants. Further, as used herein, a “biologically pure” strain is intended to mean the strain separated from materials with which it is normally associated in nature. Note that a strain associated with other strains, or with compounds or materials that it is not normally found with in nature, is still defined as “biologically pure”. A monoculture of a particular strain is, of course, “biologically pure”. As used herein, the term “enriched culture” of an isolated microbial strain refers to a microbial culture that contains more than 50%, 60%, 70%, 80%, 90%, or 95% of the isolated strain.

As used herein, a microbial consortium refers to a mixed population of two or more microbial strains. Typically, the microbial strains may form naturally or are combined together and achieve a specific purpose. For example, the microbial consortium of the present invention in combination is for biodegradation.

As used herein, the terms “organic matter” (used interchangeably with organic material) encompass any material comprising carbon including both fossilised and non-fossilised materials. Non-limiting examples of organic matter include biomass, lignocellulosic matter, and hydrocarbon-containing materials (e.g. lignite, oil shale and peat).

As used herein, “waste comprising organic matter” includes biological waste, manure, green waste, municipal waste, sewage, food and agricultural waste, and industrial organic waste. Manures can include manure produced by humans and various animals, including farm animals, such as, cows, sheep, horses, pigs, goats, rabbits, and poultry such as chickens, turkeys, and ducks. Green waste can include a variety of substrates from several sources such as yard wastes including grass clippings, tree, brush and hedge trimmings, and leaves, as well as domestic and commercial food waste. Municipal waste can include residential and commercial refuse, such as paper, wood, food and yard wastes. Waste that may be biodegraded or composted can be separated from commingled non-biodegradable matter. Sewage sludge can be used as a source of organic waste.

### DETAILED DESCRIPTION OF EXEMPLARY EMBODIMENTS

According to a first aspect, the present invention provides a method of producing a product for waste degradation. The method includes isolating a plurality of microbial strains from one or more sources of food waste as separate colonies on a solid medium; selecting a plurality of the isolated microbial strains based on size and/or abundance of colonies of each of the microbial strains, the selected microbial strains being selected based on having a larger colony size and/or a higher colony abundance compared to other microbial strains; and combining the selected microbial strains to produce a microbial consortium for waste degradation.

In particular, the colonies of each of the microbial strains may be differentiated based on size, shape and colour.

The method of producing a product for waste degradation may include assessing food degradation of microbial consortia comprising the combined microbial strains.

The selected microbial strains may be selected from a group consisting of Candida sp., Pediococcus sp. and Enterobacter cloacae. In particular, the group may consist of Candida glabrata DSMZ 32770, Pediococcus sp. DSMZ 32729, Enterobacter cloacae DSMZ 32739 and Enterobacter sp. DSMZ 32730.

The method of producing a product for waste degradation may further include culturing each of the selected microbial strains before combining the selected microbial strains. Each of the selected microbial strains may be cultured at a temperature of between about 30 degrees Celsius (° C.) and about 70° C. The step of combining the selected microbial strains may include inoculating the cultured microbial strains into a culture medium.

The method of producing a product for waste degradation may also include immobilizing the microbial consortium on a carrier. A mass ratio of an aqueous culture of the microbial consortium to the carrier may be about 2:1. The carrier may be sawdust, spent grains or derived from empty fruit bunch of oil palm.

In a second aspect, the present invention provides a product for waste degradation including a microbial consortium. The microbial consortium includes a combination of microbial strains isolated from one or more sources of food waste as separate colonies on a solid medium and selected based on size and/or abundance of colonies of each of the microbial strains, the selected microbial strains being selected based on having a larger colony size and/or a higher colony abundance compared to other microbial strains.

The colonies of each of the microbial strains may be differentiated based on size, shape and colour.

The selected microbial strains may be selected from a group consisting of Candida sp., Pediococcus sp. and Enterobacter cloacae. In particular, the group may consist of Candida glabrata DSMZ 32770, Pediococcus sp. DSMZ 32729, Enterobacter cloacae DSMZ 32739 and Enterobacter sp. DSMZ 32730. The product for waste degradation may further include a carrier, with the microbial consortium being immobilized on the carrier. The carrier may be sawdust, spent grains or derived from empty fruit bunch of oil palm.

In a third aspect, the present invention provides an isolated microbial strain selected from a group consisting of Candida sp., Pediococcus sp. and Enterobacter cloacae. In particular, the group may consist of Candida glabrata DSMZ 32770, Pediococcus sp. DSMZ 32729, Enterobacter cloacae DSMZ 32739 and Enterobacter sp. DSMZ 32730. The DSMZ numbers are the deposit numbers with Leibniz-Institute DSMZ-German Collection of Microorganisms (a Budapest treaty international deposit authority).

In a fourth aspect, the present invention also provides a substantially pure culture of any of the isolated microbial strains in accordance with the third aspect.

In a fifth aspect, the present invention provides a microbial consortium or a mixed microbial composition comprising two or more strains selected from a group consisting of Candida sp., Pediococcus sp. and Enterobacter cloacae. In particular, the group may consist of Candida glabrata DSMZ 32770, Pediococcus sp. DSMZ 32729, Enterobacter cloacae DSMZ 32739 and Enterobacter sp. DSMZ 32730.

The present invention also includes a method for preparing a substantially pure culture of an isolated strain as described herein; as well as a method of preparing a microbial consortium or mixed microbial composition as described herein.

The invention further includes a method for preparing the microbial consortium or mixed microbial composition as described herein, comprising the steps of:
(i) culturing each microbial strain separately; and
(ii) combining two or more of the cultured microbial strains to give the microbial consortium or mixed microbial composition.

Step (i) may comprise culturing each microbial strain separately to form a separate pre-inoculation culture; and step (ii) may comprise inoculating a culture medium with all the pre-inoculation cultures and culturing to give the resultant microbial consortium or mixed microbial composition.

The present invention further includes a method for preparing a microbial consortium or a mixed microbial composition, comprising the steps of:
(i) culturing each of the microbial strains Candida glabrata (DSMZ 32770), Pediococcus sp. (DSMZ 32729), Enterobacter cloacae (DSMZ 32739) and Enterobacter sp. (DSMZ 32730) to form a substantially pure culture of each microbial strain; and
(ii) combining all four substantially pure cultures to give the microbial consortium or mixed microbial composition.

Step (i) may comprise culturing each of the microbial strains Candida glabrata (DSMZ 32770), Pediococcus sp. (DSMZ 32729), Enterobacter cloacae (DSMZ 32739) and Enterobacter sp. (DSMZ 32730) to give four substantially pure pre-inoculation cultures of each microbial strain; and step (ii) may comprise inoculating a culture medium with all four pre-inoculation cultures.

The substantially pure culture, microbial consortium or a mixed microbial composition as described herein may be immobilized with a solid medium. Accordingly, a method as described herein further includes immobilizing the microbial strain, microbial consortium or mixed microbial composition with a solid medium. Advantageously, the solid medium serves as a solid support to enhance the stability of the microbial consortium for commercial usage. Any suitable solid medium may be used for immobilizing the substantially pure culture, microbial consortium or a mixed microbial composition. The solid medium is preferably cheap and biodegradable. Examples of a suitable solid medium include but are not limited to sawdust, spent grains or a solid medium derived from empty fruit bunch of oil palm.

The isolated microbial strains may be used individually or the microbial consortium or mixed microbial composition may be used for biodegradation of waste.

The present invention also provides a method for biodegrading waste comprising organic matter comprising the steps of:
(i) mixing at least one isolated microbial strain selected from the group consisting of Candida glabrata (DSMZ 32770), Pediococcus sp. (DSMZ 32729), Enterobacter cloacae (DSMZ 32739) and Enterobacter sp. (DSMZ 32730) to the waste comprising organic matter to form a mixture; and
(ii) fermenting the mixture.

The present invention further provides a method for biodegrading waste comprising organic matter comprising the steps of:
(i) mixing two or more strains selected from the group consisting of Candida glabrata (DSMZ 32770), Pediococcus sp. (DSMZ 32729), Enterobacter cloacae (DSMZ 32739) and Enterobacter sp. (DSMZ 32730) to form a mixture; and
(ii) fermenting the mixture.

The waste comprises organic matter that may be biodegraded.

In a sixth aspect, the present invention provides a waste degradation method. The waste degradation method includes: providing one of the product for waste degradation, the isolated microbial strain, the substantially pure culture of the isolated microbial strain or the microbial consortium or the mixed microbial composition; mixing the one of the product for waste degradation, the isolated microbial strain, the substantially pure culture of the isolated microbial strain or the microbial consortium or the mixed microbial composition with waste; and biodegrading the waste with the one of the product for waste degradation, the isolated microbial strain, the substantially pure culture of the isolated microbial strain or the microbial consortium or the mixed microbial composition. The waste may be biodegraded at a temperature of between about 30 degrees Celsius (° C.) and about 50° C. In an embodiment where the product for waste degradation is provided, the product for waste degradation and the waste may be provided in a mass ratio of about 1:1.

Having now generally described the invention, the same will be more readily understood through reference to the following examples which are provided by way of illustration, and are not intended to be limiting of the present invention.

### EXAMPLES

A new microbial consortium WM4 was developed by mixing the key microbial strains isolated from food waste samples from Westcom Solution Pte Ltd.

### Example 1: Isolation of Microbial Strains

Key microbial strains were isolated from food waste samples from Westcom Solution Pte Ltd. Different colonies of the same strain were largely identified based on their colony morphology, colour, shape, size and abundance on the agar plates. Strains were selected based on their size and abundance on the agar plates.

One yeast clone C1 and one bacterial clone C2 were isolated from Sample 2 and two bacterial clones C3 and C4 were isolated from Sample 3.

### Example 2: Identification of Isolated Strains

The four (4) isolates were identified in a lab and further confirmed by Leibniz-Institute DSMZ-German Collection of Microorganisms.

TABLE 1
Identity of Isolated Microbial Strains

| Sample 2 | Name of the Microbial Strain |
|---|---|
| C1 | Candida glabrata |
| | (DSMZ 32770) |
| C2 | Pediococcus sp. |
| | (DSMZ 32729) |
| Sample 3 | Name of the Bacteria |
| C3 | Enterobacter cloacae |
| | (DSMZ 32739) |
| C4 | Enterobacter sp. |
| | (DSMZ 32730) |

### Example 3: Growth and Culture Concentration of Isolates

All four (4) key strains were grown at both 30° C. and 50° C. in 100 mL liquid medium (with 5 g/L sodium chloride, 2 g/L K₂HPO₄.2H₂O, Yeast extract 10 g/L, Dextrose 50 g/L) to compare cell growths. It was found that all bacterial strains grew better at 30° C. than at 50° C. After 24 h of growth, the pH of the culture was adjusted to 7 using NaOH and an additional 50 g/L of glucose was added and the culture was incubated for another 24 h. In case of cultures at 50° C., due to low OD₆₀₀ values, thus low consumption of glucose, no further addition of glucose was conducted after 24 h. After 48 h of growth, cultures incubated at 30° C. had much higher OD₆₀₀ compared to cultures incubated at 50° C. (Table 2).

In one particular example, all ten bacterial cultures (cultivated at both temperatures) were mixed uniformly and centrifuged at 4000 rpm, 4° C. for 10 min. The culture supernatant was decanted leaving behind 200 ml medium to re-suspend the cell pellet for immobilization over saw dust. It will be appreciated that in an alternative embodiment, the four different microbial strains grown at one temperature may be combined together.

TABLE 2
OD₆₀₀ of the microbial isolates at 30° C. and 50° C.

| Sample 2 | OD₆₀₀ at 30° C. | OD₆₀₀ at 50° C. |
|---|---|---|
| C1 | 60 | 0.10 |
| C2 | 0.7 | 0.7 |
| Sample 3 | OD₆₀₀ at 30° C. | OD₆₀₀ at 50° C. |
| C3 | 3.5 | 0.01 |
| C4 | 4.0 | 0.01 |

### Example 4: Immobilization of Concentrated Cultures Over Saw Dust

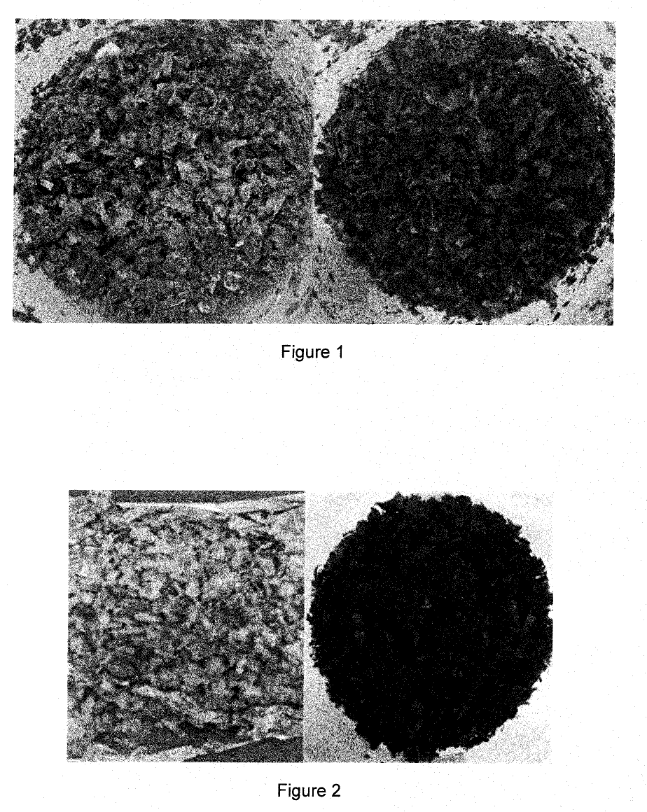

After cultivation, the microbial cells were harvested and immobilized by adding saw dust and the immobilized cells were air dried for 2-3 days in a fume hood (FIG. 1). After 72 h of air drying, the microbial consortium WM4 was ready for use for waste degradation. The moisture content of the microbial consortium WM4 was measured by drying it at 80° C. in an oven for 48 h followed by measuring its weight loss before and after the oven drying. Its moisture content was thus determined to be 32.2%.

### Example 5: Food Waste Degradation Studies

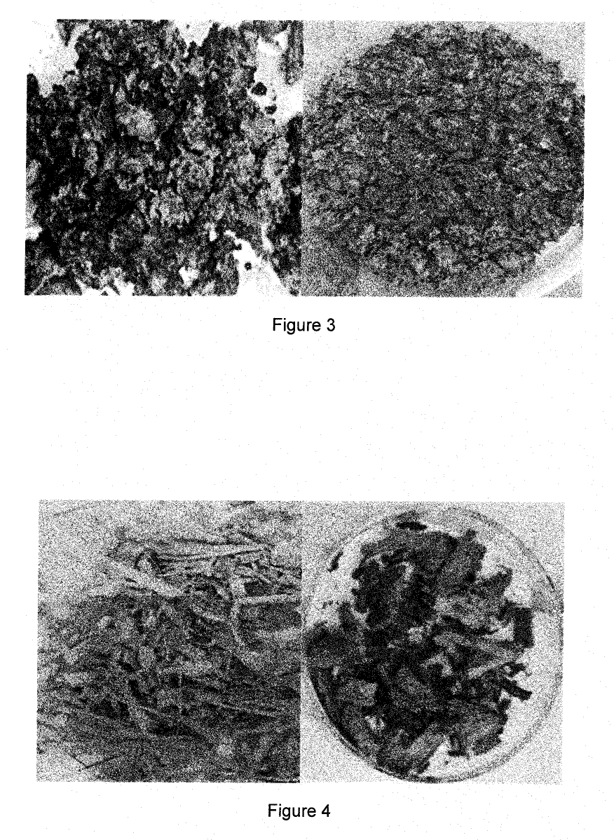

Autoclaved chicken, cooked meat and fresh fruit waste including bagasse were separately degraded using WM4. Food degradation experiments were conducted by mixing 50 g (wet weight) of the immobilized microbial cells (WM4) from Example 4 with 50 g of food waste (wet weight) and incubating the mixture at 50° C. and 200 rpm. Autoclaved chicken, waste cooked meat and fresh fruit waste including bagasse were efficiently degraded after 162 h, 114 h and 114 h, respectively (FIGS. 2 to 4). The degradation effects were at least as good as those using the commercially available microbial consortia in which even after 162 h, the un-degraded three food wastes were still obviously visible.

It will be appreciated that any one of the strains may be used to biodegrade waste comprising organic material. It will also be appreciated that a microbial consortium according to the present invention may comprise two or more of the four bacteria strains.

### Example 6: Simplified Preparation of WM4 for Easier Mass Production

A simplified process to produce a microbial consortium was developed to favour its commercial applications. Pre-inoculation cultures of each strain were prepared separately and any two, three or all four pre-cultures may be used to inoculate a culture media to culture strains to give a mixed culture (i.e. the microbial consortium).

### 6.1 Co-Cultivation of all Four Strains

All four (4) bacterial isolates except C2 were pre-cultured individually in 20 ml yeast extract peptide dextrose (YPD) broth (Yeast extract 10 g/L, Peptone 20 g/L and Dextrose 20 g/L) at 30° C. overnight, and the isolate C2 (Pediococcus sps) was cultivated in 20 ml of De Man, Rogosa and Sharpe (MRS) broth (10 g/L Proteose peptone, 10 g/L Beef extract, 5 g/L Yeast extract, Dextrose 20 g/L, Polysorbate 80 1 g/L, Ammonium citrate 2 g/L, Sodium acetate 5 g/L, Magnesium sulphate 0.1 g/L, Manganese sulphate 0.050 g/L, Dipotassium phosphate 2 g/L) and incubated over night at 30° C. Next day, all four (4) cultures were inoculated into a conical flask containing 300 mL liquid medium with (5 g/L sodium chloride, 2 g/L K₂HPO₄.2H₂O, Yeast extract 10 g/L, Dextrose 50 g/L) to give a final culture volume of 380 ml with an initial OD₆₀₀ of 1.97. The mixture was incubated at 30° C. for 24 h when the OD₆₀₀ was measured to be 18.9 and pH was 4.0. To neutralize the acidity, 2.5 g of NaHCO₃ was added to a final pH of 7, then an additional 50 g/L of glucose was added and the culture was incubated for another 24 h. The final OD₆₀₀ reached 40 at 48 h.

### 6.2 Immobilization of Co-Cultivated Strains Over Saw Dust

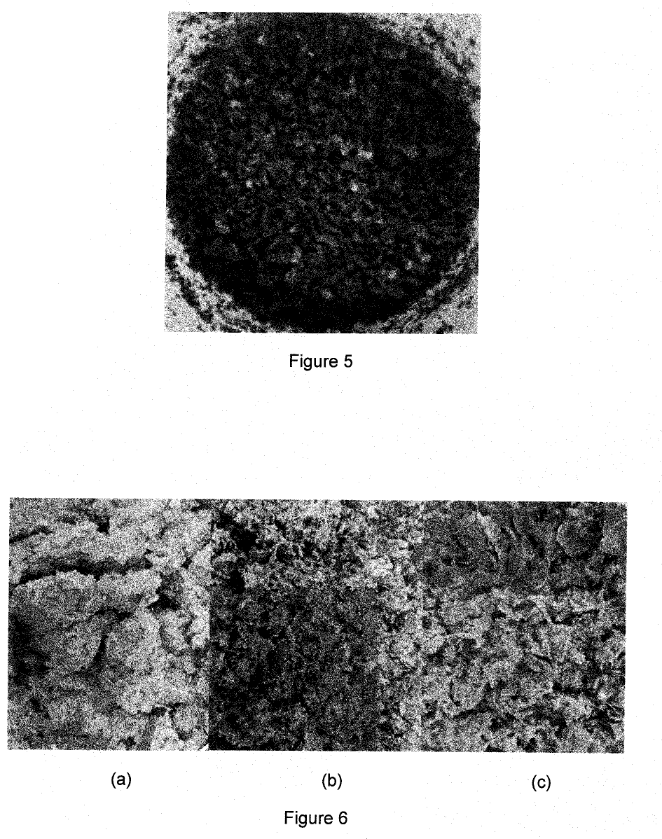

Saw dust was added to the harvested co-culture to immobilize the microbial consortium. After air drying of this immobilized microbial consortium, the microbial consortium WM4 was ready for use for waste degradation. The moisture content of the immobilized microbial consortium (WM4) was calculated by drying at 80° C. in an oven for 48 h followed by measuring its weight loss before and after the oven drying. Its moisture content was thus determined to be 61.2%. FIG. 5 shows the microbial consortium WM4 prepared by co-cultivation of four (4) strains followed by immobilization with saw dust.

### 6.3 Food Waste Degradation

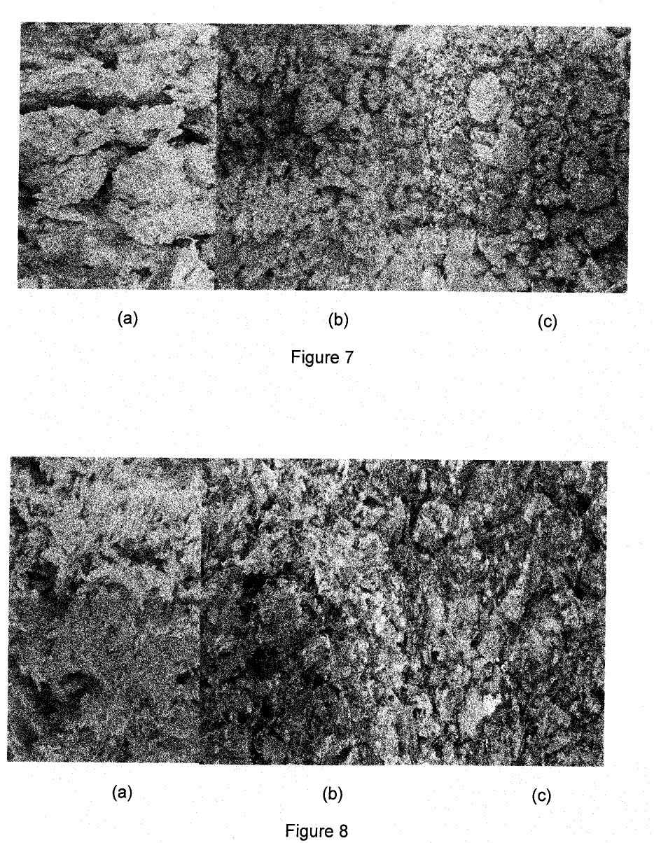

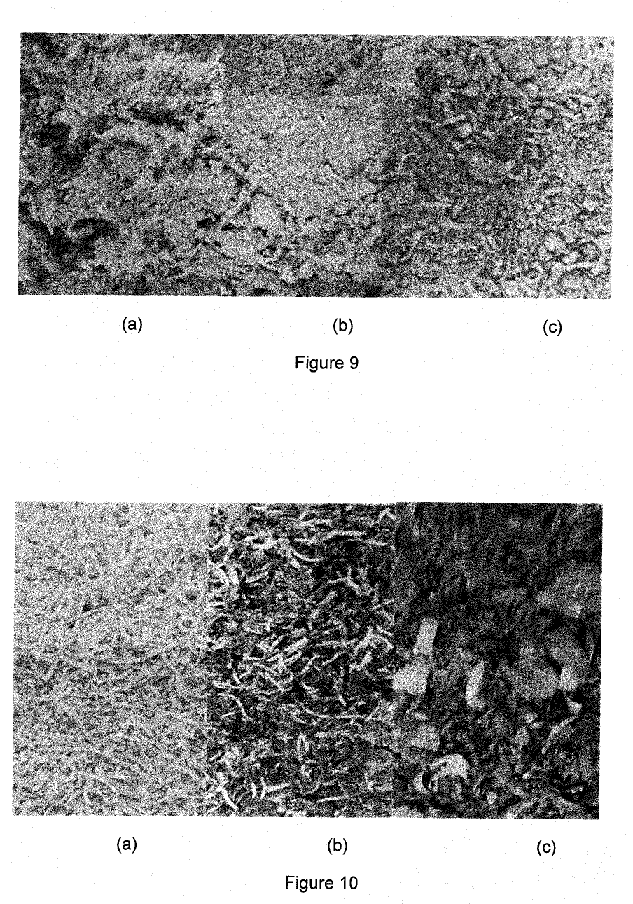

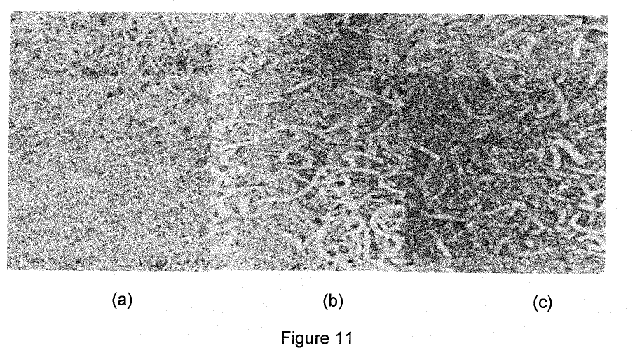

The immobilized consortium (WM4) prepared from Example 6.2 above and commercial microbial consortium and food wastes (cooked meat and noodles) were used in 1:1 (w/w, wet weight) ratio. 50 g of the immobilized consortium (WM4) prepared from Example 6.2 above and commercial microbial consortium (50 g, wet weight) used as a control were mixed separately with 50 g of food waste (wet weight) and incubated at 37° C. with shaking at 200 rpm. Cooked meat (50 g, wet weight), raw noodles (50 g, wet weight) (Hiap Giap Food Manufacture Pte Ltd) and the combination of cooked meat and raw noodles (25 g for each, wet weight) were tested as substrates for food waste degradation (FIGS. 6-11).

### 6.4 Total Solid Reduction and Food Waste Reduction

Total solid (food waste+immobilized microbial consortia) weight loss and food waste weight loss using the immobilized consortium (WM4) prepared from Example 6.2 above and commercial microbial consortium were measured. Ten gram of each of the different food samples (test and control) from Example 6.3 was taken and re-suspended in 50 ml of water. Each sample was thoroughly vortexed for 2-3 min and centrifuged at 4000 rpm for 30 min. The particulate matter and water-insoluble substances of each of the different food samples were pelleted down in a separate falcon tube for each sample and the soluble fractions in supernatant were decanted. The falcon tubes with pellet were dried at 80° C. until no weight loss was observed. The total solid reduction and food waste reduction were calculated after degradation for 48 h (for meat and meat plus noodles) and 120 h (for noodles alone), respectively (Table 3).

The formula for calculating total solid reduction is:

$$\begin{matrix}Total solid reduction ( \% ) = \frac{{A}_{i} - {A}_{f}}{{A}_{i}} \times 100 \% \\ {A}_{i} initial dry weight of food waste and microbial consortium\end{matrix}$$

$${A}_{f} final dry weight of food waste and microbial consortium after degradation$$

The formula for calculating food waste reduction is:

$$Food waste reduction ( \% ) = \frac{{A}_{f} - {M}_{i}}{{F}_{i}}$$

$${A}_{f} final dry weight of food waste and microbial consortium after degradation$$

$${M}_{i} initial dry weight of microbial consortium$$

$${F}_{i} initial dry weight of food waste$$

TABLE 3
Total solid reduction and total food waste reduction after
degradation

| | | Total solid | Food waste |
|---|---|---|---|
| No. | Sample name | reduction (%) | reduction (%) |
| 1 | Noodles and commercial | 25.2 | 61.1 |
| | microbial consortium | | |
| 2 | Noodles and WM4 | 31 | 86 |
| 3 | Meat and commercial microbial | 6 | 20.9 |
| | consortium | | |
| 4 | Meat and WM4 | 36.3 | 91.2 |
| 5 | Meat, Noodles and commercial | 8.9 | 33.9 |
| | microbial consortium | | |
| 6 | Meat, noodles and WM4 | 35 | 79.4 |

It was seen that for noodles only samples, after 120 h, the total solid reduction and food waste reduction by WM4 were respectively 1.2 times and 1.4 times higher than the commercial microbial consortium. In the case of the meat only samples, after 48 h, the total solid reduction and food waste reduction by WM4 were respectively 6.0 times and 4.4 times higher than the commercial microbial consortium. In the case of the combination of meat and noodles samples, after 48 h, the total solid reduction and food waste reduction by WM4 were 3.9 times and 2.3 times higher, respectively. Therefore, MW4 is much more efficient than the commercial microbial consortium for food waste degradation in terms of its much higher degradation degree and lower operating temperature (<40° C.). It is postulated that the degradation time was shorter for the meat and noodle sample compared to the noodle only sample because the presence of meat provided more nitrogen for growth of the microbial strains.

### 6.5 Measurement of pH of Degraded Food Wastes

Solid samples (1.0 g, wet weight) taken from each of the food waste samples before and after degradation were separately added to 10 ml of tap water. The degradation times were 120 h for noodles and 48 h for meat alone and meat and noodle samples. Each mixture was vortexed and kept at room temperature for the solid to settle down. Then the pH of the supernatant from each sample was measured (Table 4).

TABLE 4
pH changes of solid samples before and after degradation (1 g
dissolved in 10 ml water)

| | | pH (before | pH (After |
|---|---|---|---|
| No. | Sample name | degradation) | degradation) |
| 1 | Noodles and commercial | 6.5 | 7 |
| | microbial consortium | | |
| 2 | Noodles and WM4 | 6.5 | 7 |
| 3 | Meat and commercial microbial | 6.5 | 4 |
| | consortium | | |
| 4 | Meat and WM4 | 5.5 | 5 |
| 5 | Meat, Noodles and commercial | 6.5 | 6 |
| | microbial consortium | | |
| 6 | Meat + noodles + WM4 | 6.5 | 5 |

It was noticed that in case of noodle degradation, there was an increase in pH using both WM4 and the commercial microbial consortium after the degradation. In the cases of cooked meat and combination of cooked meat and noodles, a decrease in pH was observed. HPLC analyses of the degradation products from the noodles, meat alone and meat+noodles (WM4 and controls) showed the presence of larger amount of acetic acid and smaller amount of butyric acid as the major acidic substances.

### Example 7 Discussion/Conclusion

A new microbial consortium WM4 was developed by isolating the microbes from the food waste products collected from Westcom Solution Pte Ltd and combining the most powerful isolated ones to form the new microbial consortium.

Three (3) bacterial strains (Enterobacter sps, Pediococcus sps, Enterobacter cloacae) and one (1) yeast strain of Candida glabrata were isolated from the food waste products and all of them are classified under Biosafety Level 1 (BSL 1).

The cultivation conditions were optimized and the microbial consortium was immobilized on cheap and biodegradable solid medium to enhance the stability of the microbes for commercial usage.

Mixing the individual cultures of all four (4) isolates grown at 30° C. followed by harvesting and adding saw dust to immobilize the cells gave the microbial consortium WM4, which is able to degrade food wastes at 30-50° C. more efficiently than a commercially available microbial consortium.

A simplified process for preparing WM4 was developed in which the four (4) isolates were co-cultivated and then immobilized on saw dust to reduce the production cost and favor its commercial applications.

The new microbial consortium WM4 was able to convert food wastes more efficiently than a commercially available microbial consortium in terms of shortened treatment time and higher degradation degree of food wastes.

The new microbial consortium WM4 works more efficiently than a commercially available microbial consortium in degrading food waste to organic fertilizer, which saves the treatment time and energy consumption and improves the process efficiency favoring the process economy.

Food waste treatment using a commercially available microbial consortium takes a longer time (over 24 h per cycle) and needs a higher temperature (50° C.) for the treatment. Lower degradation rate and higher energy consumption were observed using this commercially available microbial consortium for food waste treatment. The new microbial consortium WM4 can work more efficiently than the commercially available microbial consortium and can be operated at lower temperatures (below 40° C.), significantly improving the process efficiency and economy.

WM4 degraded cooked meat, noodles and the combination of cooked meat and noodles more efficiently than a commercial microbial consortium, leading to the formation of a more acidic environment.

While preferred embodiments of the invention have been illustrated and described, it will be clear that the invention is not limited to these embodiments only. Numerous modifications, changes, variations, substitutions and equivalents will be apparent to those skilled in the art without departing from the scope of the invention as described in the claims.

Further, unless the context clearly requires otherwise, throughout the description and the claims, the words “comprise”, “comprising” and the like are to be construed in an inclusive as opposed to an exclusive or exhaustive sense; that is to say, in the sense of “including, but not limited to”.

1. A method of producing a product for waste degradation, comprising:
isolating a plurality of microbial strains from one or more sources of food waste as separate colonies on a solid medium;
selecting a plurality of the isolated microbial strains based on size and/or abundance of colonies of each of the microbial strains, wherein the selected microbial strains are selected based on having a larger colony size and/or a higher colony abundance compared to other microbial strains; and
combining the selected microbial strains to produce a microbial consortium for waste degradation.

2. The method according to claim 1, wherein the colonies of each of the microbial strains are differentiated based on size, shape, and color.

3. The method according to claim 1, further comprising assessing food degradation of microbial consortia comprising the combined microbial strains.

4. The method according to claim 1, wherein the selected microbial strains are selected from a group consisting of Candida sp., Pediococcus sp. and Enterobacter cloacae.

5. The method of claim 1, wherein the selected microbial strains are selected from a group consisting of Candida glabrata DSMZ 32770, Pediococcus sp. DSMZ 32729, Enterobacter cloacae DSMZ 32739 and Enterobacter sp. DSMZ 32730.

6. The method according to claim 1, further comprising culturing each of the selected microbial strains before combining the selected microbial strains.

7. The method according to claim 6, wherein each of the selected microbial strains is cultured at a temperature of between about 30 degrees Celsius (° C.) and about 70° C.

8. The method according to claim 6, wherein the step of combining the selected microbial strains comprises inoculating the cultured microbial strains into a culture medium.

9. The method according to claim 1, further comprising immobilizing the microbial consortium on a carrier.

10. The method according to claim 9, wherein the carrier is sawdust, spent grains or derived from empty fruit bunch of oil palm.

11. A product for waste degradation, comprising:
a microbial consortium comprising a combination of microbial strains isolated from one or more sources of food waste as separate colonies on a solid medium and selected based on size and/or abundance of colonies of each of the microbial strains, wherein the selected microbial strains are selected based on having a larger colony size and/or a higher colony abundance compared to other microbial strains.

12. The product for waste degradation according to claim 11, wherein the colonies of each of the microbial strains are differentiated based on size, shape, and color.

13. The product for waste degradation according to claim 11, wherein the microbial strains are selected from a group consisting of Candida sp., Pediococcus sp. and Enterobacter cloacae.

14. The product for waste degradation according to claim 11, wherein the microbial strains are selected from a group consisting of Candida glabrata DSMZ 32770, Pediococcus sp. DSMZ 32729, Enterobacter cloacae DSMZ 32739 and Enterobacter sp. DSMZ 32730.

15. The product for waste degradation according to claim 11, further comprising a carrier, wherein the microbial consortium is immobilized on the carrier.

16. The product for waste degradation according to claim 15, wherein the carrier is sawdust, spent grains or derived from empty fruit bunch of oil palm.

17. An isolated microbial strain selected from a group consisting of Candida sp., Pediococcus sp. and Enterobacter cloacae.

18. The isolated microbial strain according to claim 17, wherein the isolated microbial strain is selected from a group consisting of Candida glabrata DSMZ 32770, Pediococcus sp. DSMZ 32729, Enterobacter cloacae DSMZ 32739 and Enterobacter sp. DSMZ 32730.

19. A substantially pure culture of the isolated microbial strain according to claim 17.

20. The substantially pure culture of the isolated microbial strain according to claim 19 immobilized with a solid medium.

21. The substantially pure culture of the isolated microbial strain according to claim 20, wherein the solid medium is sawdust, spent grains or derived from empty fruit bunch of oil palm.

22. A microbial consortium or a mixed microbial composition comprising two or more microbial strains selected from a group consisting of Candida sp., Pediococcus sp. and Enterobacter cloacae.

23. The microbial consortium or the mixed microbial composition according to claim 22, wherein the two or more microbial strains are selected from a group consisting of Candida glabrata DSMZ 32770, Pediococcus sp. DSMZ 32729, Enterobacter cloacae DSMZ 32739 and Enterobacter sp. DSMZ 32730.

24. The microbial consortium or the mixed microbial composition according to claim 22 immobilized with a solid medium.

25. The microbial consortium or the mixed microbial composition according to claim 24, wherein the solid medium is sawdust, spent grains or derived from empty fruit bunch of oil palm.

26. A waste degradation method, comprising:
providing one of a product for waste degradation, an isolated microbial strain, a substantially pure culture of the isolated microbial strain, and a microbial consortium or a mixed microbial composition;
mixing the one of the product for waste degradation, the isolated microbial strain, the substantially pure culture of the isolated microbial strain or the microbial consortium or the mixed microbial composition with waste; and
biodegrading the waste with the one of the product for waste degradation, the isolated microbial strain, the substantially pure culture of the isolated microbial strain or the microbial consortium or the mixed microbial composition,
wherein:
the product for waste degradation comprises a microbial consortium comprising a combination of microbial strains isolated from one or more sources of food waste as separate colonies on a solid medium and selected based on size and/or abundance of colonies of each of the microbial strains, wherein the selected microbial strains are selected based on having a larger colony size and/or a higher colony abundance compared to other microbial strains;
the isolated microbial strain is selected from a group consisting of Candida sp., Pediococcus sp. and Enterobacter cloacae; and
the microbial consortium or the mixed microbial composition comprises two or more microbial strains selected from a group consisting of Candida sp., Pediococcus sp. and Enterobacter cloacae.

27. The waste degradation method according to claim 26, wherein the waste is biodegraded at a temperature from about 30 degrees Celsius (° C.) to about 50° C.

28. The waste degradation method according to claim 26, wherein the product for waste degradation is provided and wherein the product for waste degradation and the waste are provided in a mass ratio of about 1:1.
